(19) United States
(12) Patent Application Publication
(10) Pub. No.: US 2014/0163354 A1
(43) Pub. Date: Jun. 12, 2014

(54) MRI/CAT SCAN PATIENT ARM SUPPORT

(71) Applicant: Andrew J. Breth, Holdingford, MN (US)

(72) Inventor: Andrew J. Breth, Holdingford, MN (US)

(21) Appl. No.: 13/712,763

(22) Filed: Dec. 12, 2012

(51) Int. Cl. A61B 5/055

(52) U.S. Cl. CPC A61B 5/0555
USPC 600/415

(57) ABSTRACT

A MRI/CAT scan patient arm support assembly comprising a lower frame including a pair of horizontal side members positioned parallel to each other, at least one cross member connecting the pair of horizontal side members, the pair of horizontal side members and the at least one cross member forming a MRI/CAT scan bed-engaging surface, an upper frame connected to and extending angularly upward from the lower frame, the upper frame having a pair of vertical side members positioned parallel to each other, a handle interconnecting the pair of vertical side members, the handle oriented in a substantially horizontal manner and positioned above the lower frame, the handle vertically adjustable along the vertical side members, and at least one removable armrest connected to one of the vertical side members of the upper frame, the removable armrest rotatably adjustable about the vertical side member and adjustable along the vertical side member.

### CROSS REFERENCE TO RELATED APPLICATIONS

I hereby claim benefit under Title 35, United States Code, Section 120 of U.S. patent application Ser. No. 12/813,504; filed Jun. 11, 2010. This application is a continuation-in-part of the Ser. No. 12/813,504 application. The Ser. No. 12/813,504 application is currently pending. The Ser. No. 12/813,504 application is hereby incorporated by reference into this application.

### STATEMENT REGARDING FEDERALLY SPONSORED RESEARCH OR DEVELOPMENT

Not applicable to this application.

### BACKGROUND OF THE INVENTION

1. Field of the Invention

The present invention relates generally to a patient positioning device and more specifically it relates to a patient arm support for efficiently maintaining a patient's arms beyond them during medical procedures, such as during MRI, CAT scan tomography (computerized axial) and IV (intravenous contrast) applications.

2. Description of the Related Art

Any discussion of the related art throughout the specification should in no way be considered as an admission that such related art is widely known or forms part of common general knowledge in the field.

Known methods of positioning a patient's arms overhead during a CT (computed tomography) or MERI scan involve the patient holding their arms overhead for prolonged periods. The patient must immobilize their arm, to hold it steady for the scan, which can often times result in much discomfort. Additionally, for IV applications, the arms must generally be held perfectly straight and still, which can be physically difficult to maintain without the occurrence of an infiltrated vein from patient movement.

Among other nuisances, these difficulties cause delays in the medical exams and may cause errors which may lead to repeated exams or actual medical problems and pain experienced by the patient, such as when the IV becomes dislodged because patients cannot hold their arms in the required positions. Because of the inherent problems with the related art, there is a need for a new and improved patient arm support for efficiently maintaining a patient's arms beyond them during medical procedures, such as during MRI, CAT scan tomography (computerized axial) and IV (intravenous contrast) applications.

### BRIEF SUMMARY OF THE INVENTION

A system for efficiently maintaining a patient's arms beyond them during medical procedures, such as during MRI, CAT scan tomography (computerized axial) and IV (intravenous contrast) applications. The invention generally relates to patient positioning device which includes a lower frame, an upper frame pivotally connected to the lower frame, wherein the upper frame extends angularly upward from a forward end of the lower frame, and an adjustment means interconnecting the lower frame with the upper frame. The adjustment means is adapted to secure the upper frame in a plurality of angularly upward positions relative the lower frame. The adjustment means may be comprised of a biasing adjustment member or a U-shaped brace. The upper frame may also include a plurality of armrests.

There has thus been outlined, rather broadly, some of the features of the invention in order that the detailed description thereof may be better understood, and in order that the present contribution to the art may be better appreciated. There are additional features of the invention that will be described hereinafter and that will form the subject matter of the claims appended hereto. In this respect, before explaining at least one embodiment of the invention in detail, it is to be understood that the invention is not limited in its application to the details of construction or to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 is an upper perspective view of the present invention;

FIG. 2 is a side view of the present invention in use with the patient outside of the scanning area;

FIG. 3 is a side view of the present invention in use with the patient moving to within the scanning area;

FIG. 4 is a side sectional view of the present invention illustrating the patient grabbing onto one of the handles;

FIG. 5 is a side view of the present invention illustrating the armrest being rotated;

FIG. 6 is a magnified front sectional view of the arm. rest aligned with the support brackets;

FIG. 7 is a magnified front sectional view of the armrest connected to the support brackets;

FIG. 8 is an upper perspective view of an alternate embodiment of the present invention having a first adjustment means;

FIG. 9 is an upper perspective view of an alternate embodiment of the present invention having a second adjustment means;

FIG. 10 is a sectional view of the alternate embodiment of the present invention having the second adjustment means;

FIG. 11 is a side view of the alternate embodiment of the present invention having the first adjustment means showing the upper frame in a first angular position;

FIG. 12 is a side view of the alternate embodiment of the present invention having the first adjustment means showing the upper frame in a second angular position;

FIG. 13 is a perspective view showing an alternative embodiment of a MRI/CAT scan patient arm support assembly;

FIG. 13A shows a close up cross-sectional view of a first bracket of FIG. 13;

FIG. 13B shows a close up view of the removable armrest of FIG. 13;

FIG. 14 shows MRI/CAT scan patient arm support assembly positioned for use on a curved-shaped MRI/CAT scan bed of a MRI/CAT scan machine;

FIG. 15 shows a MRI/CAT scan patient arm support assembly having a mesh screen;

FIG. 16 shows a MRI/CAT scan patient arm support assembly having a removable head foam block pillow; and

FIG. 17 shows a MRI/CAT scan patient arm support assembly having a headrest-receiving member and a weighted end portion.

### DETAILED DESCRIPTION OF THE INVENTION

### A. Overview

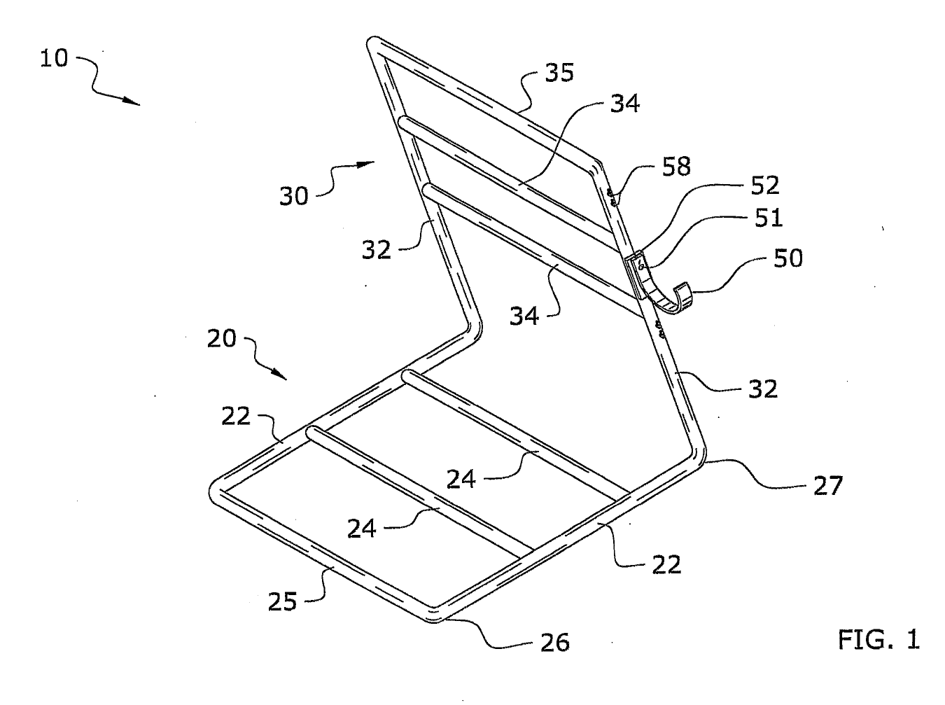

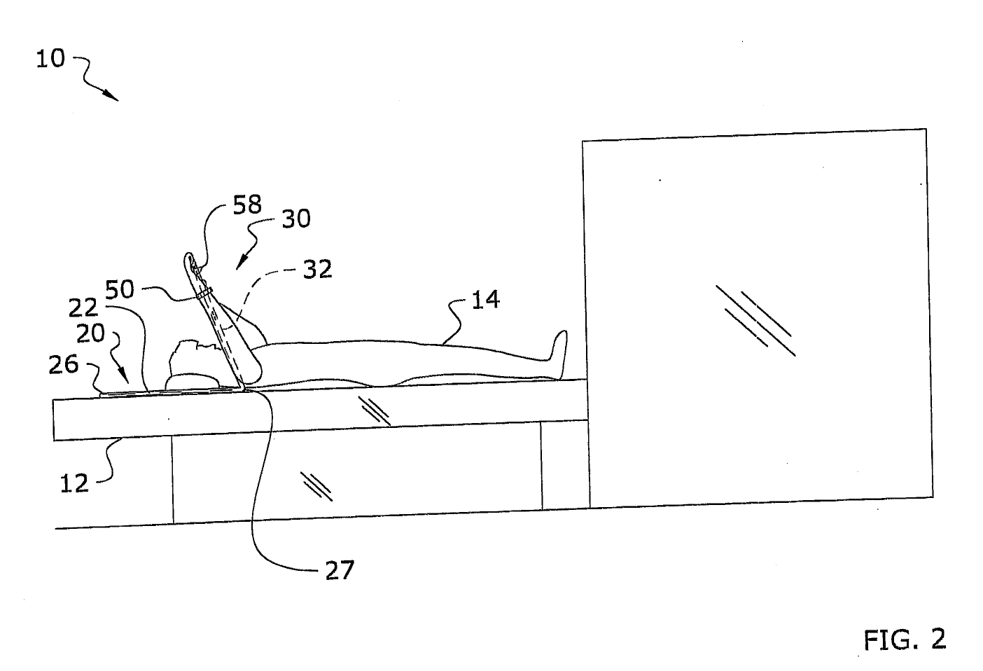

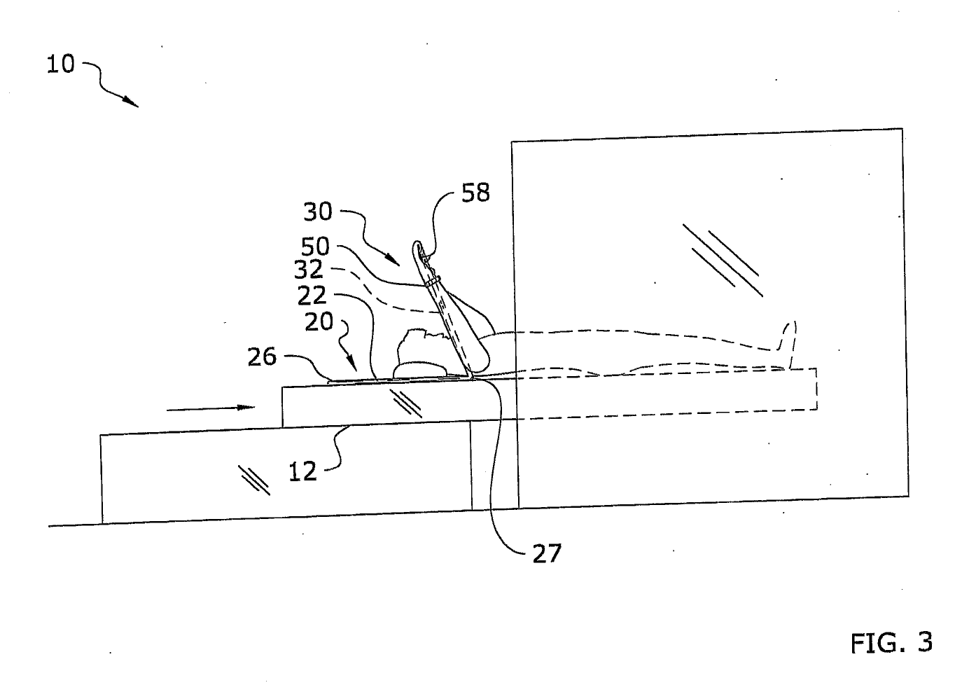

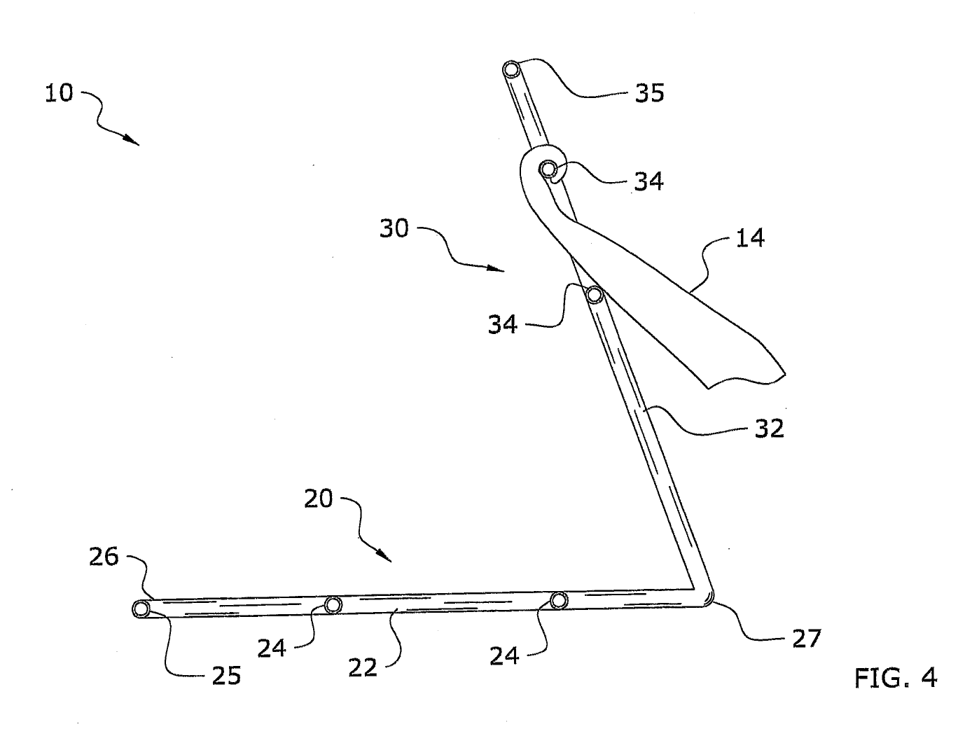

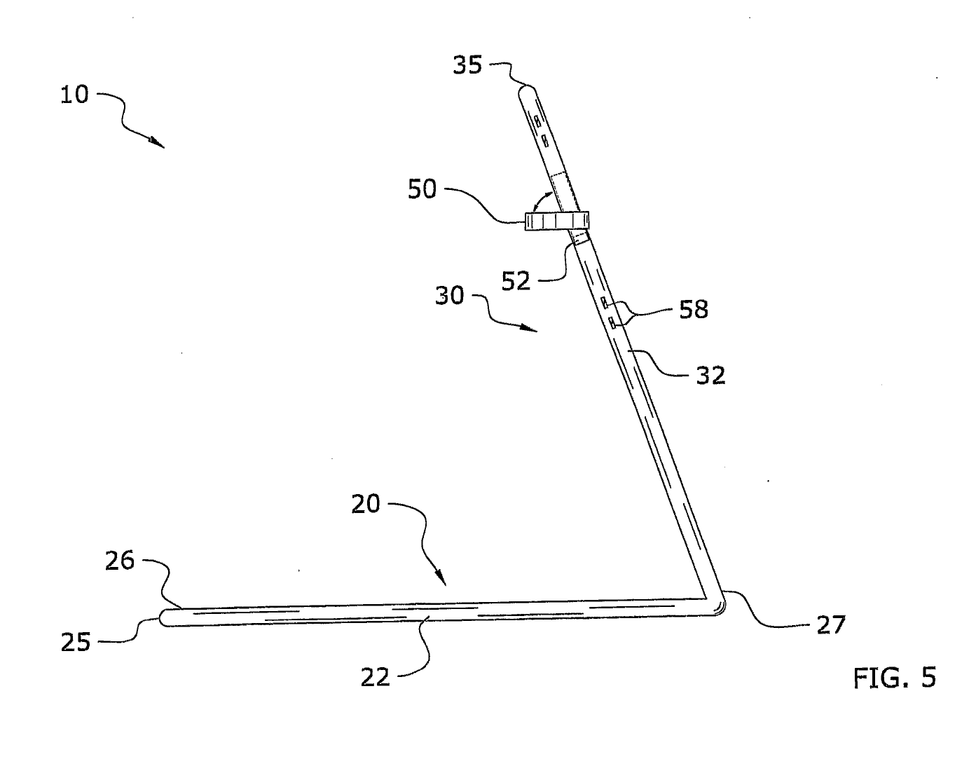

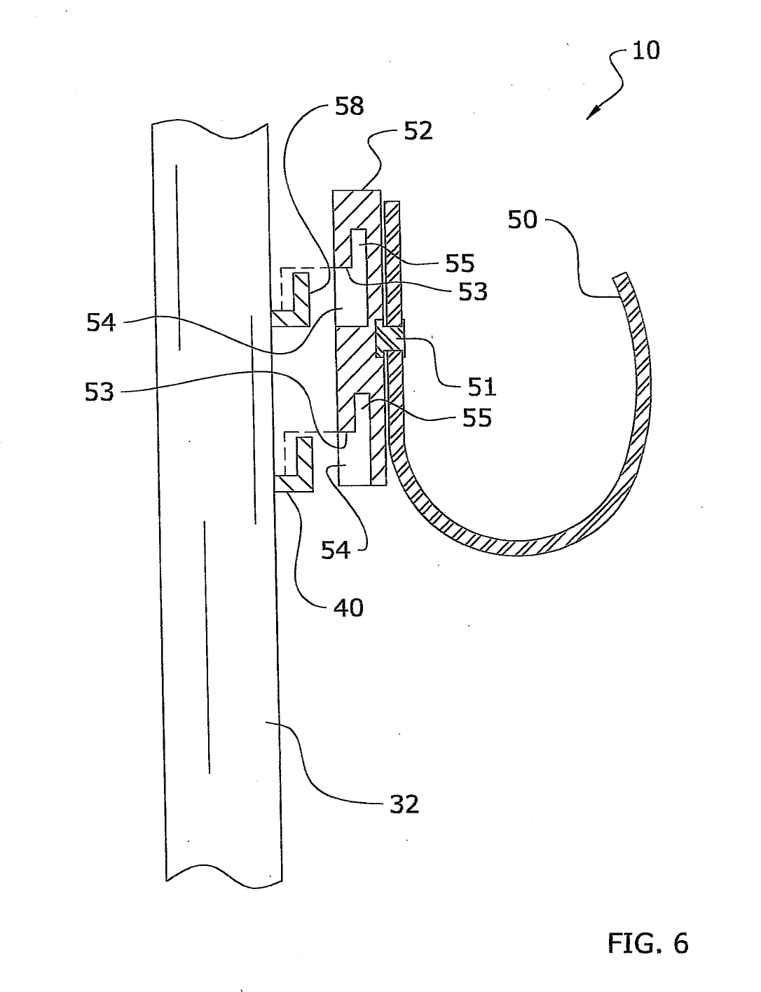

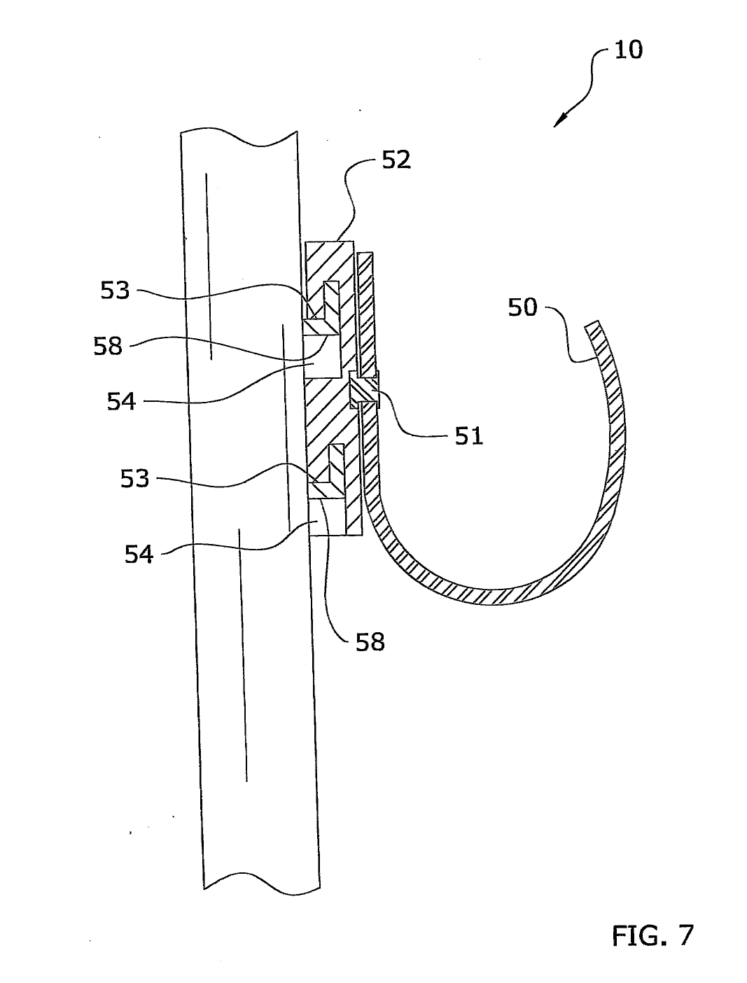

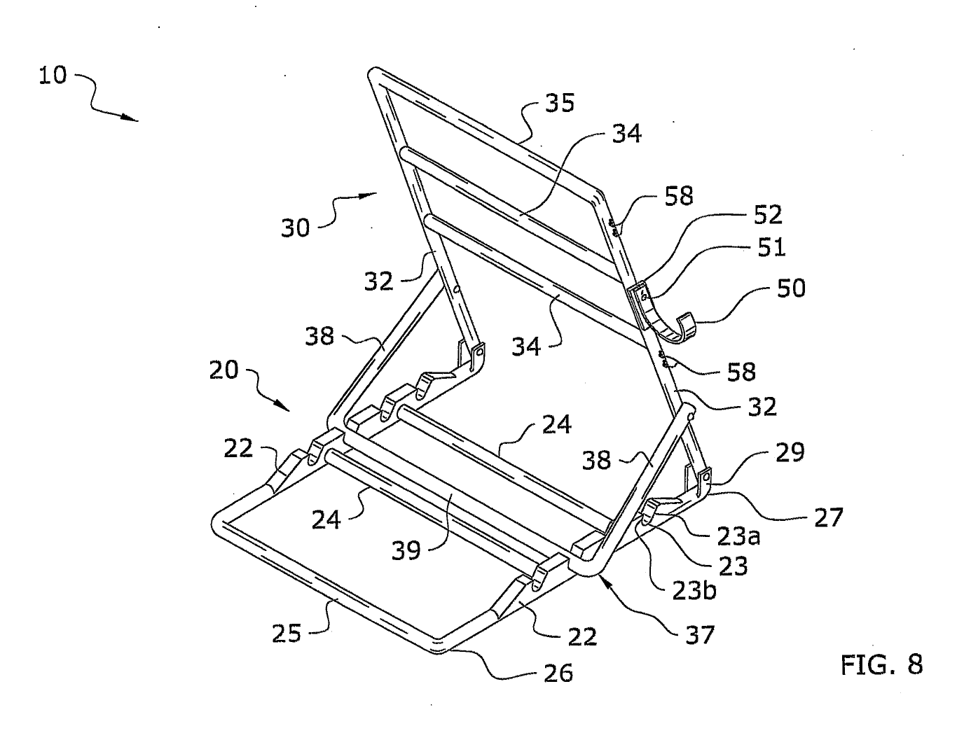

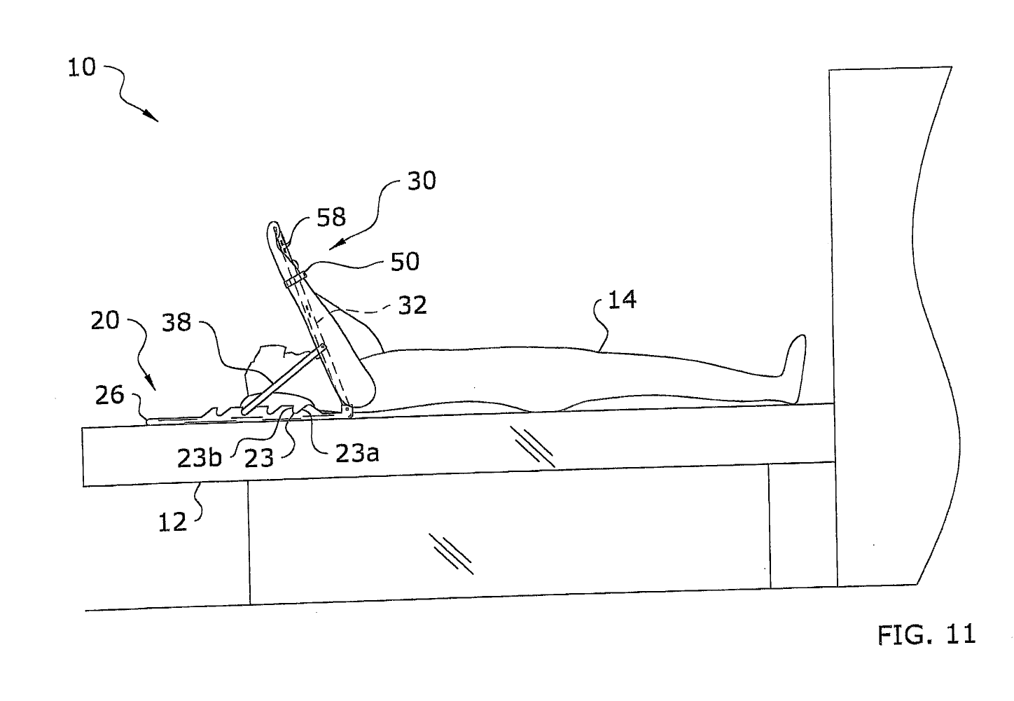

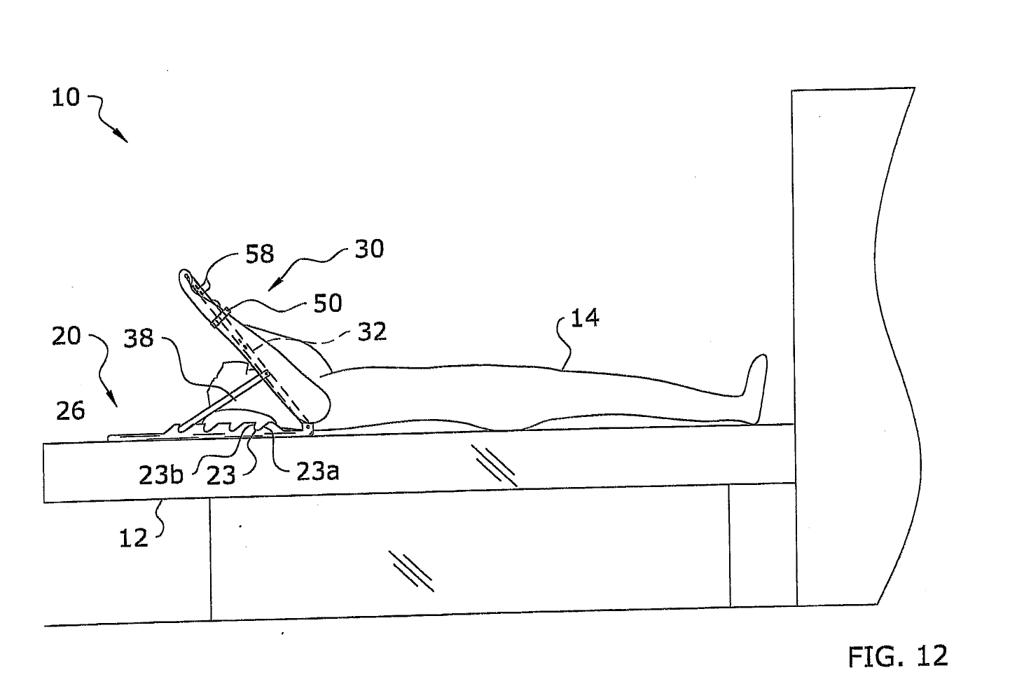

Turning now descriptively to the drawings, in which similar reference characters denote similar elements throughout the several views, FIGS. 1 through 12 illustrate a patient arm support 10, which comprises a lower frame 20 for being positioned upon a flat table 12 surface, an upper frame 30 vertically extending from a forward end 27 of the lower frame 20, wherein the upper frame 30 is supported in an upright manner via the lower frame 20, wherein the upper frame 30 includes at least one handle 34 oriented in a horizontal manner, wherein the at least one handle 34 is positioned above the lower frame 20, and a pair of arm rests 50 extending from each side of the upper frame 30. Adjustment means for pivoting the upper frame 30 about the lower frame 20 are also described.

The lower frame 20, upper frame 30, and other components of the present invention are preferably comprised of a stainless steel, aluminum, plastic, or other type of non-magnetic material that is suitable for MRI, CT, or other radioactive applications.

### B. Lower Frame

The lower frame 20 generally rests upon a flat horizontal surface, such as an exam table 12 used in MRI, CT, or IV medical procedures. It is appreciated that the lower frame 20 may be configured to be attached in a vertical manner for upright procedures. In the preferred embodiment the lower frame 20 extends sufficiently rearward and has an adequate amount of weight to maintain the upper frame 30 in an upright position without the aid of additional support devices. The lower frame 20 is thus configured so that the present invention is comprised of a self-supporting structure. The lower frame 20 may be attached to the exam table 12 or may be loosely positioned upon thereof, wherein the configuration of the lower frame 20, even when loosely positioned, is designed to maintain the upper frame 30 in an upright position.

In the preferred embodiment, the lower frame 20 includes a pair of side members 22 extending along the sides of the lower frame 20, a plurality of cross members 24 extending across and between the two side members 22 as illustrated in the figures and including an end member 25 along a rearward end 26 (opposite attachment of the upper frame 30) of the lower frame 20 and a plurality of intermittently or regularly spaced cross members 24 along the length of the side members 22 to both provide weight and support to the lower frame 20 and present invention. The cross members 24 are further preferably parallel to each other and perpendicularly positioned with respect to the side members 22.

The side members 22, the cross members 24, and end member 25 may be comprised of an integral structure or a plurality of separate structure affixed together. The side members 22, the cross members 24, and end member 25 may also be comprised of cylindrical shaped configurations, to define a hollow interior space, may be solidly configured, or may be configured in various other shapes and sizes. In addition, the lower frame 20 is preferably configured to extend along a common horizontal plane, wherein the entire lower frame 20 rests flat upon the exam table 12 or adjacent surface.

### C. Upper Frame

The upper frame 30 vertically extends from the lower frame 20 adjacent a forward end 27 of the lower frame 20. The upper frame 30 is used as a support in which the patient 14 may rest their arms or grab onto while the patient 14 is undergoing certain medical procedures. The upper frame 30 may be integrally formed with the lower frame 20 or comprised of a separate structure affixed thereto. The upper frame 30 is may be positioned perpendicularly with respect to the lower frame 20 and is supported in an upright manner via the lower frame 20. In the preferred embodiment, the upper frame 30 and the lower frame 20 define an acute angle, wherein the upper frame 30 is pivoted slightly rearward to form about a 75-degree angle with the lower frame 20.

In the preferred embodiment, the upper frame 30 includes a pair of horizontal side members 32 extending along the sides of the upper frame 30 and a plurality of handles 34 extending across and between the two side members 32 as illustrated in the figures. The handles 34 preferably include a top handle 35 along a top end (opposite attachment of the lower frame 20) of the upper frame 30 and a plurality of intermittently or regularly spaced handles 34 along the length of the side members 32 to provide multiple height locations in which the patient 14 may grab onto.

The patient 14 is preferably able to grab onto any of the vertical bars as required by the medical procedure or most comfortable 12 for the patient 14. It is appreciated that more or less handles 34 may be used with the upper frame 30 to provide the patient 14 with additionally choices on which bar to grasp, hold onto, or rest their arms upon. The handles 34 are further preferably parallel to each other and perpendicularly positioned with respect to the side members 32.

The side members 32, the handles 34 including the top handle 35 may also be comprised of cylindrical shaped configurations, to define a hollow interior space, may be solidly configured, or may be configured in various other shapes and sizes. In addition, the upper frame 30 is preferably configured to extend along a common vertical plane and preferably an angled plane slightly offset from vertical.

### D. Support Brackets

A plurality of support brackets 40 are also preferably positioned along the side members 32 of the upper frame 30. The support brackets 58 are used to receive the armrest 50. The support brackets 58 are positioned at multiple vertical locations to position the forearm at multiple vertical locations of the patient's 14 choosing. The support brackets 58 are preferably located along each of the side members 32 on each side of the upper frame 30 and are spaced evenly with accordance of the other side. Thus, the support brackets 58 upon each side of the upper frame 30 mirror each other so that a pair of arm rests 50 may likewise be positioned at similar height locations.

The support brackets 58 are positioned at multiple height locations along the vertical side members 32, and at each height location are preferably positioned in pairs to securely receive the armrest 50. Utilizing a pair of support brackets 58 (rather than one) at each height location, along each side, helps to prevent the arm rest 50 from swaying or moving while the patient's 14 arms are positioned within. Again, the multiple pairs of support brackets 58 are positioned at a plurality of height locations along each side member 32. The support brackets 58 may further be located at similar height locations as the handles 34 or differing height locations.

Each of the support brackets 58 is further preferably comprised of an L-shaped hook configuration and extends outwardly, away from the handles 34, from the side members 32. It is appreciated that alternate types of support brackets 58 may be used with the present invention rather than those described herein.

The plurality of support brackets 58 include a first set of support brackets attached to a first side of said upper frame 30 and a second set of support brackets attached to a second side of said upper frame 30. The first set and second set of support brackets 58 may further include an upper, middle, and lower set to position the arm rests 50 along the upper, middle area, or lower part of the upper frame 30 as desired.

### E. Arm Rests

The arm rests 50 are for the patient 14 to rest their arms within while undergoing various types of medical procedures, such as but not limited to MRIs, CTs, or IVs. The armrests 50 are generally used when the patient 14 must retain their arms in a set position, such as during an IV procedure. The armrests 50 are preferably adjustable 12 in height along the side members 32, wherein the forearms cuffs attach to the support brackets 58. The present invention includes a pair of armrests 50, one on each side member 32, to attach to the respective support brackets 58. It is appreciated that the present invention may include a plurality of armrests 50 on each side of the arm support 10.

In the preferred embodiment, the arm rests 50 each include a base 52 located along a rear side of the arm rests 50 and a pair of receiver slots 53 integrally extending from the base 52. The receiver slots 53 are received by the L-shaped support brackets 58 along the side members 32 to allow for easy adjustability of the arm rests 50 to a desired height location. The receiver slots 53 further mimic the shape of the support brackets 58. In the preferred embodiment, an entrance opening to within the armrest 50 is preferably substantially parallel with the orientation of the lower frame 20 and thus straight behind the patient 14.

The receiver slots 53 are further preferably only open to a rear of the base 52, wherein the sides of the base 52 do not provide access to the receiver slots as illustrated in FIG. 1. The receiver slots 53 are thus used by inserting the support brackets 58 perpendicularly within the receiver slots 53 and then lowering the base 52 so that the brackets 58 will extend upwardly within the slots 53.

The receiver slots 53 each include an opening portion 54 adjacent and interconnecting with an outer edge of the base 52 and a passageway 55 extending vertically upwardly from the opening 54 of the receiver slot 53. When inserting the support bracket 58 within the receiver slot 53, the support bracket 58 is fully inserted within the opening 54 and vertically extended within the passageway 55 by moving the base 52 downwards relative the upper frame 30.

A connector 51 secures the armrest 50 to the base 52. The connector 51 preferably allows the arm rest 50 to rotate relative the base 52 thus allowing the patient 14 to position the arm rest 50 in an orientation comfortable to them and their body position.

The arm rests 50 are preferably comprised of a semi-circular shaped configuration, such as resembling a cuff, to correlate with the shape of the patient's 14 arm so that their arm may be comfortably received within. The patient 14 further preferably positions their forearm within the respective armrest 50, wherein the arm rests 50 are configured to receive the forearm portion of the patient's 14 arms. It is appreciated that the armrests 50 may be comprised of various other shapes and configuration rather than those described herein. The arm rests 50 are further preferably comprised of a comfortable 12 material for the patient 14 to position their forearm or other part of their arm within. In an alternate embodiment, the support brackets 58 may be omitted (or simply not used).

### F. Adjustment Means

Various adjustment means may be utilized with the present invention to adjust the upwardly angular position of the upper frame 30 relative the lower frame 20. Like, the other components of the present invention, the adjustment means is comprised of a non-magnetic material thus being able to be used in various medical scanning equipment. By adjusting the angular position of the upper frame 30 about the lower frame 20, the user is able to accommodate the device 10 to correlate with the size of the individual, the orientation of the individual, the size of the medical device, or various other criteria. Various hinge means 29 may be utilized to pivotally connect the lower frame 20 to the upper frame 30. Preferably, a hinge 29 is utilized between each of the vertical side members 32 and the respective horizontal side members 22.

In one embodiment, a pivotal brace 37 is used to secure the upper frame 30 in an angular position relative the lower frame 20. The brace 37 is generally pivotally connected to the upper frame 30 and operably engages the lower frame 20. The brace 37 is also generally comprised of a U-shaped structure.

The brace 37 generally includes a pair of side portions 38 each pivotally connected to a respective vertical side member 32 of the upper frame 30 and a cross portion 39 interconnecting the side portions 38 at a lower end of the brace 37. The cross portion 39 is selectively received within correlating notches 23 of the horizontal side members 22 to retain the brace 37 and upper frame 30 in a pivotal position.

Each of the side members 22 generally include a series of spaced apart notches 23 for receiving the pivotal brace 37 and thus being able to adjust the angular position of the upper frame 30 relative the lower frame 20. Each of the notches 23 generally include a forwardly slanting forward edge 23a and a forwardly slanting rearward edge 23b to ensure that the cross portion 29 of the brace 37 is retained within the respective notches 23 when rearward pressure is applied upon the upper frame 30.

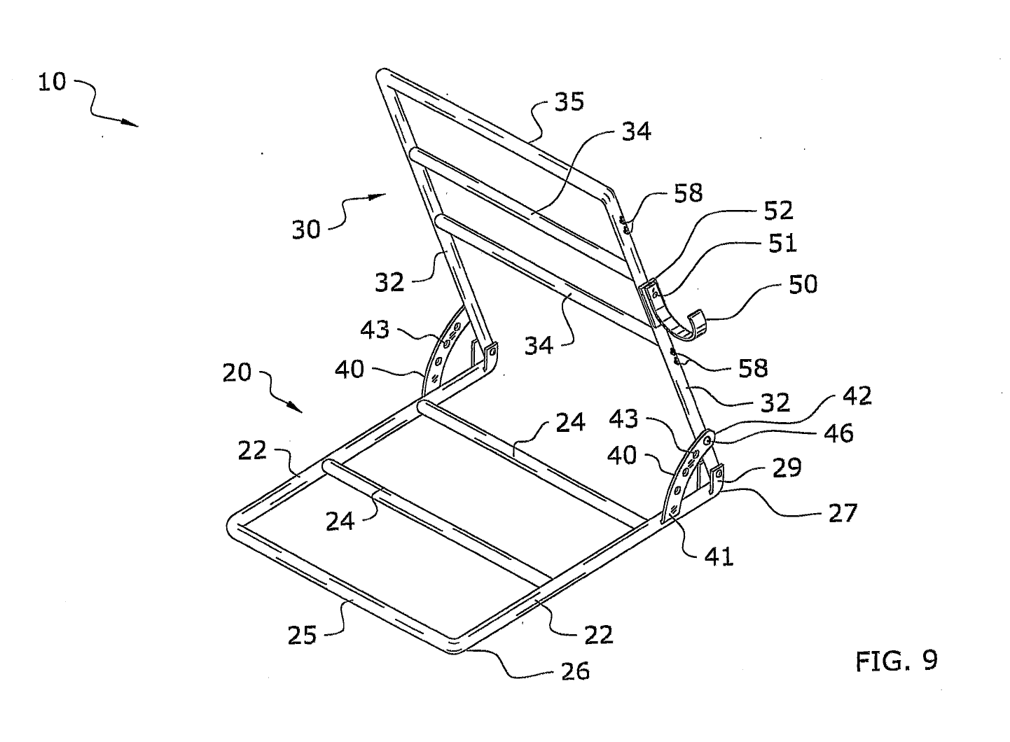

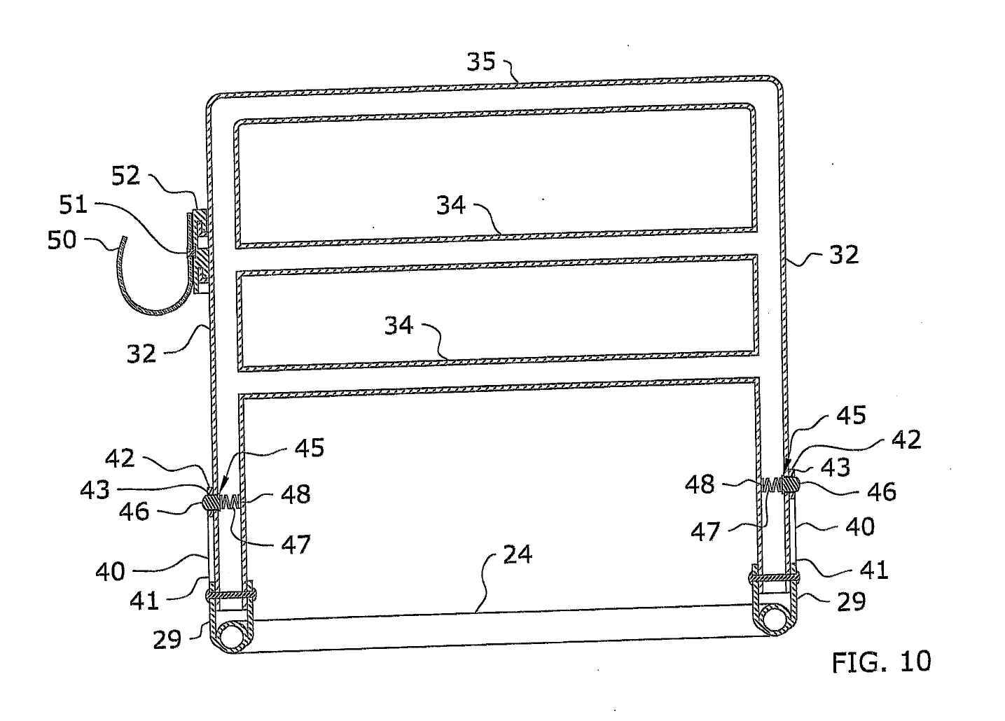

Another embodiment of the adjustment means is shown in FIGS. 9 and 10. The embodiment generally includes a corner support 40 extending from the forward end 27 of the side members 22 of the lower frame. The corner support 40 is generally relatively fixed in position. The corner support 40 also generally curves or arches toward the upper frame 30, wherein the first end 41 of the corner support 40 is attached to the lower frame 30 slightly behind the forward most end of the lower frame 30 and the second end 42 of the corner support 40 is positioned upwardly and forwardly of the first end 41. The corner support 40 may also be semi-circled or quad-circled in structure.

The corner support 40 also generally includes a plurality of openings 43 spaced apart along a linear and curved path of the corner support 40 for selectively receiving a respective adjustment member 45. It is appreciated that each side of the lower frame 20 generally includes a corner support 40 for receiving a respective adjustment member 45 thus providing rigid support to each side of the upper frame 30.

The adjustment member 45 is connected to the upper frame 30 and specifically to a respective vertical side member 32 of the upper frame 30. The adjustment member 45 generally laterally adjusts via a bias member 47 preferably comprised of a compression spring positioned within the hollow vertical side member 32. A plate support 48 is also generally secured to an inner end of the bias member 47 to retain the bias member in positioned within the upper frame.

Extending from the outer end of the bias member 48 is a protruding member 46. The protruding member 46 extends through an opening in the vertical side member 22 and then through a respective opening 43 in the cross support 40. By operably pushing the protruding member 46 inwardly past the cross support 40, the upper frame is able to pivotally adjust via the lower frame 20. When the protruding member 46 of the adjustment member 45 reaches the next opening 43, the bias member 47 causes the protruding member 46 to extend outwardly though the currently aligned opening 43 of the corner support 40 to secure the upper frame 30 in an angular position relative the lower frame 20.

### G. Operation of Preferred Embodiment

In use, the patient 14 generally lies on their back on the scanner or exam table 12 and the forward end 27 of the arm support is positioned in front of the patient 14 facing their head. The arm support 10 may be positioned upon the exam table 12 along with the patient 14 or another adjacent surface. The patient 14 may also utilize a pillow placed upon the exam table 12 under the handles 34 of the upper frame 30 adjacent the lower frame 20 if desired.

If the patient 14 is instructed to raise their arms upwards, above their head, behind them, or otherwise, the patient 14 may grasp onto any one of the handles 34 of the upper frame 30 or may rest their arms within the arm rests 50 along the sides of the upper frame 30. The vertical height location of the arm rests 50 may be vertically adjusted by simply lifting the base 52 upwards so that the support brackets 58 may be removed from the receiver slots 53. For example, if the patient 14 desires to raise their arms to a higher location, the base 52 is removed from the support brackets 58 and positioned upon a set of support brackets above the previously utilized support brackets. The support brackets at the higher location are now inserted within the receiver slots 53. Likewise, when the patient desires to needs to lower their arras, the base 52 is removed from the currently used support brackets.

The support brackets at a lower location are now inserted within the base 52 so that the armrest is located at a lower location with respect to a previous location of the armrests 50. It is appreciated that the present invention may be utilized in other applications, rather than medical procedures, that require an individual to rest their arms behind them or overhead while lying on a table 12. The patient may also adjust the angular position of the upper frame 30 relative the lower frame 20 via the adjustment means.

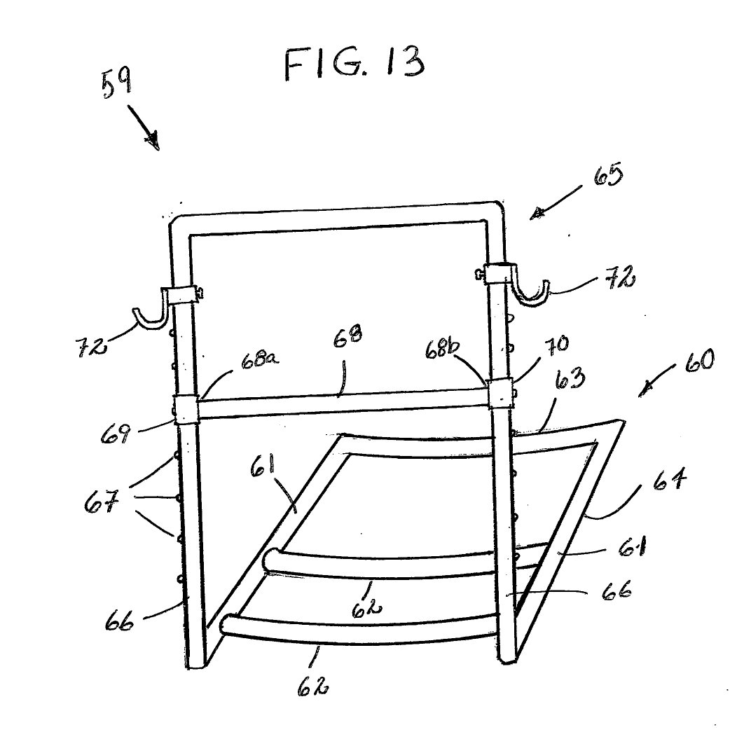

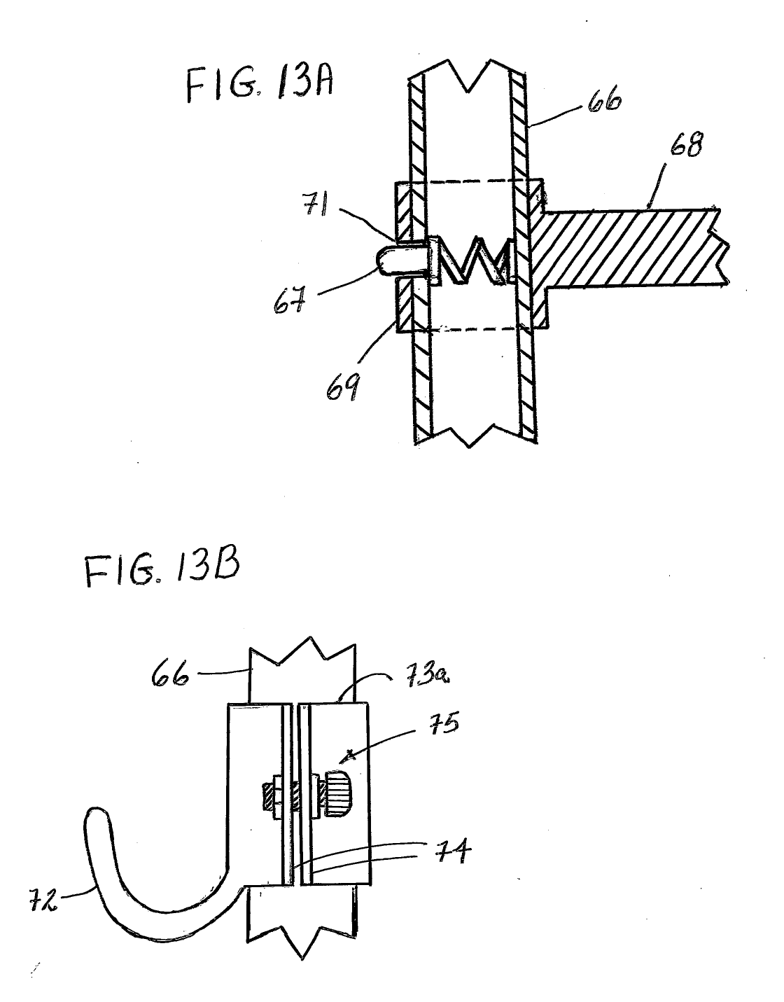

FIG. 13 is a perspective view showing an alternative embodiment of a MRI/CAT scan patient arm support assembly 59 of the present invention.

Similar to the embodiment of the patient arm support of FIGS. 1-5, MRI/CAT scan patient arm support assembly 59 generally comprises a lower frame 60 having a pair of horizontal side members 61 shown positioned parallel to each other. Although the lower frame may include from as few as one to a plurality of cross members, lower frame 60 is shown having a pair of cross member, and preferably curved shaped cross members 62 and a curved shaped end member 63 connecting the pair of horizontal side members 61. A feature of MRI/CAT scan patient arm support assembly 59 is that the pair of horizontal side members 61, the cross members 62, and end member 63 connect to form a MRI/CAT scan bed-engaging surface 64 whether the MRI/CAT scan bed-engaging surface 64 is flat or more commonly curved shaped.

MRI/CAT scan patient arm support assembly 59 also includes an upper frame 65 connected to and extending angularly upward from lower frame 60. Upper frame 65 comprises a pair of vertical side members 66 positioned parallel to each other. In the embodiment of FIG. 13 each vertical side member 66 is shown including a plurality of spring loaded pins 67 extending out from vertical side member 66 and spaced preferably linearly along vertical side member 66 with the positioning or location of the plurality of spring loaded pins 67 on each of the vertical side members 66 corresponding to each other.

Upper frame 65 also includes a handle 68 interconnecting the pair of vertical side members 66 with handle 68 oriented in a substantially horizontal manner and positioned above lower frame 60. A feature of handle 68 is that handle 68 is vertically adjustable along the vertical side members 66 permitting a customize fit of MRI/CAT scan patient arm support assembly 59 based on the size and shape of the patient during use of MRI/CAT scan patient arm support assembly 59

In the embodiment of FIG. 13, handle 68 is shown having a first bracket 69 located at a first end 68a of handle 68 and a second bracket 70 located at a second end 68b of handle 68. Brackets 69 and 70 function to connect handle 68 to the pair of vertical side members 66.

Referring to FIG. 13A, which shows a close up cross-sectional view of bracket 69 of FIG. 13 which has similar parts to bracket 70, bracket 69 includes a orifice 71 for receiving one of the spring loaded pins 67 therethrough. The engagement of spring-loaded pin 67 into orifice 71 functions to lock bracket 69 to a specific location on vertical side member 66 proximal the location of spring-loaded pin 67. To unlock bracket 69 from the specific location on vertical side member 66 a user presses spring-loaded pin 67 into horizontal member 66 and moves orifice 71 of bracket 69 away from spring-loaded pin 67.

MRI/CAT scan patient arm support assembly 59 also includes at least one removable armrest connected to one of the vertical side members 66 and preferably a removable armrest 72 connected to each of the vertical side members 66 of the upper frame 65. A feature of removable armrest 72 is that removable armrest 72 is rotatably adjustable about or around the vertical side member 66 and adjustable along the vertical side member 66 that the removable armrest 72 is connected to thereby permitting adjustability of MRI/CAT scan patient arm support assembly 59 based on the size and shape of the patient during use of MRI/CAT scan patient arm support assembly 59.

Referring to FIG. 13B, which shows a close up view of the removable armrest 72 of FIG. 13, removable armrest 72 includes a clam-shaped body 73 having an interior surface 73a for engagement with a surface of horizontal member 66. Clam-shaped body 73 includes a set of corresponding lips 74, which when moved closer to each other eventually brings the interior surface 73a of the body 73 into engagement with the surface of horizontal member 66. Located on the set of corresponding lips 74 of clam-shaped body 73 is a nut and bolt securement system 75 which function to maintain the securement of clam-shaped body 73 to horizontal member 66. Once the interior surface 73a of the body 73 engages the surface of horizontal member 66, nut and bolt securement system 75 operate to move corresponding lips 74 further towards each other which eventually will prevent movement of armrest 72 with respect to vertical side member 66 due to frictional forces created by the engagement of their surface. The hold of clam-shaped body 73 on horizontal member 66 may be released by movement of corresponding lips 74 away from each other.

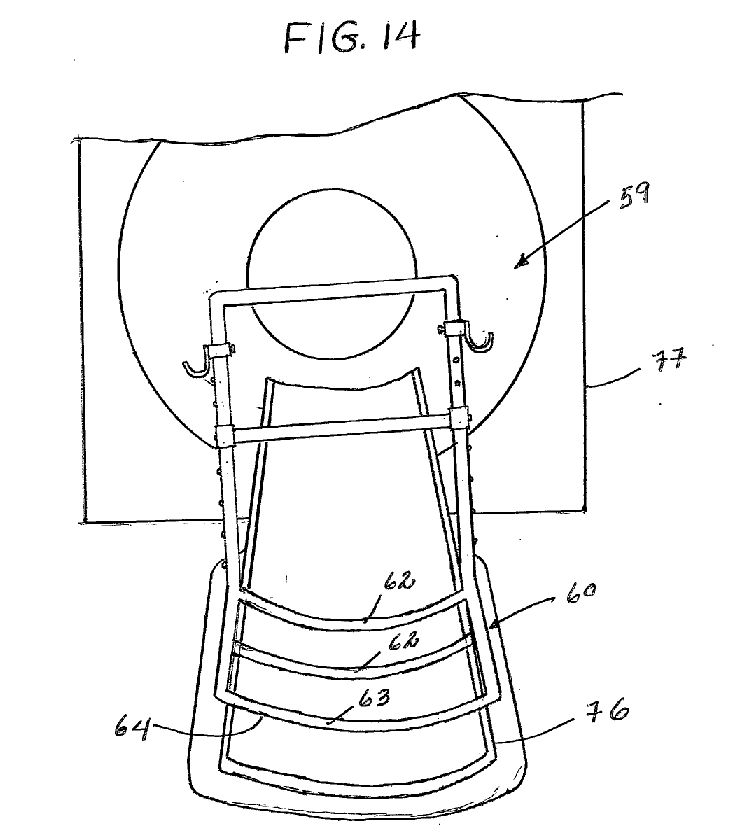

FIG. 14 shows MRI/CAT scan patient arm support assembly 59 positioned for use on a curved-shaped MRI/CAT scan bed 76 of a MRI/CAT scan machine 77. Note that the curved shaped cross members 62 and curved shaped end member 63 connecting the pair of horizontal side members 61 conforms to the curvature of the curved-shaped MRI/CAT scan bed 76 of a MRI/CAT scan machine 77 to increase balance and stability of MRI/CAT scan patient arm support assembly 59 compared to devices that have linear-shaped cross members and linear-shaped shaped end member for their lower frame.

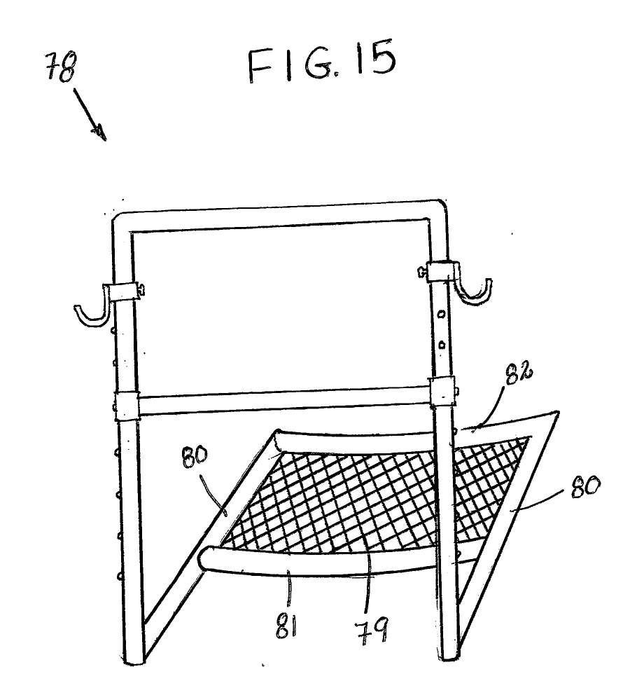

FIG. 15 shows an alternative embodiment of a MRI/CAT scan patient arm support assembly 78 having similar components to the MRI/CAT scan patient arm support assembly 59 of FIGS. 13 and 14. However MRI/CAT scan patient arm support assembly 78 further includes a mesh screen 79 supported by a pair of horizontal side members 80, a curved-shaped cross member 81 and a curve-shaped end member 82.

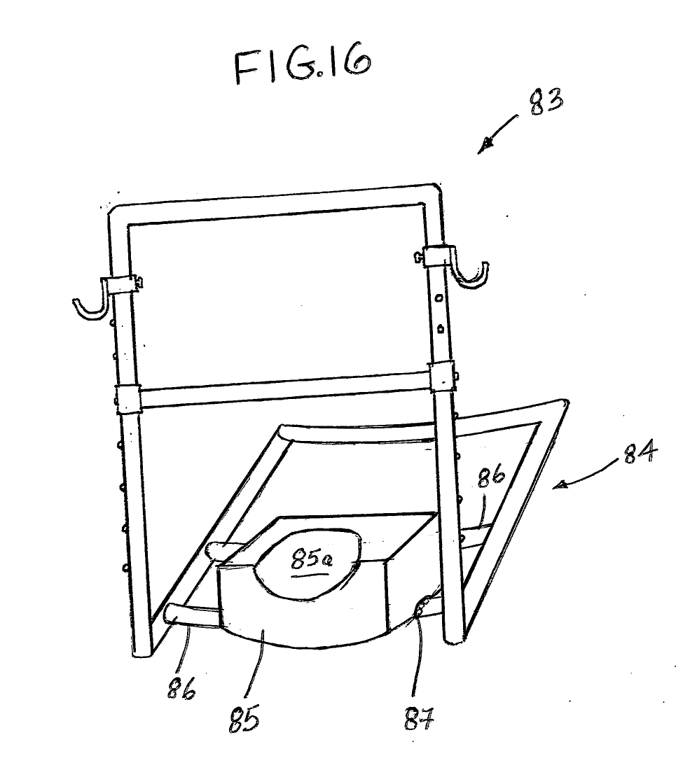

FIG. 16 shows an alternative embodiment of a MRI/CAT scan patient arm support assembly 83 having similar components, to the MRI/CAT scan patient arm support assembly 59 of FIGS. 13 and 14. However a lower frame 84 of MRI/CAT scan patient arm support assembly 83 includes a removable head foam block pillow 85 having a patient headrest region 85a secured to lower frame 84 and more specifically, the curved-shaped cross members 86 by a hook and loop fasteners system 87, which is commonly known by the trade name Velcro® system.

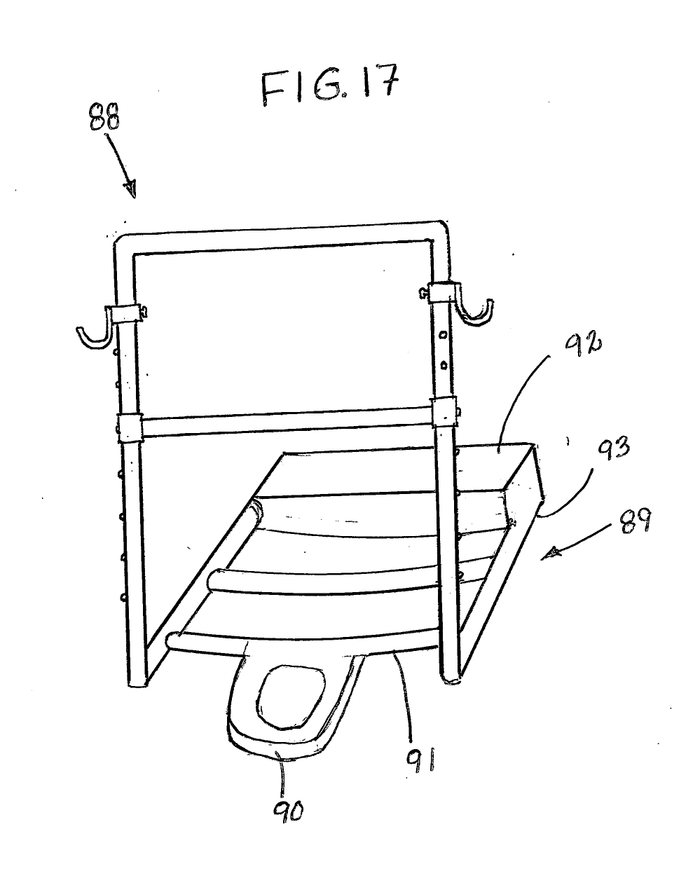

FIG. 17 shows an alternative embodiment of a MRI/CAT scan patient arm support assembly 88 having similar components to the MRI/CAT scan patient arm support assembly 59 of FIGS. 13 and 14. However a lower frame 89 of MRI/CAT scan patient arm support assembly 88 includes a headrest-receiving member 90 extending from a cross member 91. Headrest-receiving member 90 may be used to support a patient's head thereon as-is or preferably for receiving a pillow or various other types of headrests thereon.

The lower frame 89 of MRI/CAT scan patient arm support assembly 88 also is shown including a weighted end portion 92 located proximal an end member 93 of lower frame 89. Weighted end portion 92 functions to provide MRI/CAT scan patient arm support assembly 88 with stability and balance during use.

Unless otherwise defined, all technical and scientific terms used herein have the same meaning as commonly understood by one of ordinary skill in the art to which this invention belongs. Although methods and materials similar to or equivalent to those described herein can be used in the practice or testing of the present invention, suitable methods and materials are described above. All publications, patent applications, patents, and other references mentioned herein are incorporated by reference in their entirety to the extent allowed by applicable law and regulations. In case of conflict, the present specification, including definitions, will control. The present invention may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

I claim:

1. A MRI/CAT scan patient arm support assembly comprising:
a lower frame including a pair of horizontal side members positioned parallel to each other;
at least one cross member connecting said pair of horizontal side members, said pair of horizontal side members and said at least one cross member forming a MRI/CAT scan bed-engaging surface;
an upper frame connected to and extending angularly upward from said lower frame, said upper frame having a pair of vertical side members positioned parallel to each other;
a handle interconnecting said pair of vertical side members, said handle oriented in a substantially horizontal manner and positioned above said lower frame, said handle vertically adjustable along said vertical side members; and
at least one removable armrest connected to one of said vertical side members of said upper frame, said removable armrest rotatably adjustable about said vertical side member and adjustable along said vertical side member.

2. The MRI/CAT scan patient arm support assembly of claim 1 wherein said at least one cross member of said lower frame is curved-shaped to conform to a curve shaped surface of a MRI/CAT scan bed.

3. The MRI/CAT scan patient arm support assembly of claim 1 wherein said pair of vertical side members each including a plurality of spring loaded pins extending from and spaced along said vertical side members; and
wherein said handle of said upper frame including a first bracket located at a first end of said handle and a second bracket located at a second end of said handle, said brackets connecting said handle to said pair of said vertical side members, said brackets each having a orifice for receiving one of said spring loaded pins therethrough to lock said bracket to a specific location on said vertical side member.

4. The MRI/CAT scan patient arm support assembly of claim 1 wherein said removable armrest includes a clam-shaped body having corresponding lips and an interior surface for engaging a surface of said horizontal member, and further including a nut and bolt securement system located on said lip of clam-shaped body.

5. The MRI/CAT scan patient arm support assembly of claim 4 wherein each of said vertical side members include said removable armrest connected thereto.

6. The MRI/CAT scan patient arm support assembly of claim 1 wherein said lower frame includes a mesh screen supported by said pair of horizontal side members and said at least one cross member.

7. The MRI/CAT scan patient arm support assembly of claim 1 wherein said lower frame includes a removable head foam block pillow having a patient headrest region fixedly secured to said lower frame by a hook and loop fasteners system.

8. The MRI/CAT scan patient arm support assembly of claim 1 wherein said lower frame includes a headrest-receiving member extending from said cross member.

9. The MRI/CAT scan patient arm support assembly of claim 1 wherein said lower frame includes a weighted end portion, said weighted end portion providing said lower frame with stability and balance in use.

10. The MRI/CAT scan patient arm support assembly of claim 1 including a hinge means pivotally connecting said pair of horizontal side members of said lower frame to said pair of vertical side members of said upper frame; and
an adjustment means interconnecting said lower frame with said upper frame, said adjustment means adapted to secure said upper frame in a plurality of angularly positions relative said lower frame.

11. The MRI/CAT scan patient arm support assembly of claim 10 wherein said adjustment means includes at least one corner support extending from a forward end of said lower frame, said at least one corner support having a plurality of spaced openings that follows a curved radial path; and
at least one adjustment member extending from said vertical side member of said upper frame and including a compression spring for providing a biasing force to said at least one adjustment member, said adjustment member interconnecting said upper frame with said corner support and operably extending within an opening of said series of spaced openings relative a pivotal orientation of said upper frame.

12. The MRI/CAT scan patient arm support assembly of claim 10 wherein said adjustment means includes a U-shaped brace having side portions pivotally connected to each of said vertical side members of said upper frame and a cross portion to operably engage each of said horizontal side member of said lower frame; and
wherein each of said horizontal side members of said lower frame includes a series of notches to selectively receive said cross portion of said U-shaped brace when adjusting said angularly position of said upper frame.

13. A MRI/CAT scan patient arm support assembly comprising:
a lower frame including a pair of horizontal side members positioned parallel to each other;
at least one cross member connecting said pair of horizontal side members, said pair of horizontal side members and said at least one cross member forming a MRI/CAT scan bed-engaging surface;
an upper frame connected to and extending angularly upward from said lower frame, said upper frame having a pair of vertical side members positioned parallel to each other, said pair of vertical side members each including a plurality of spring loaded pins extending from and spaced along said vertical side members;
a handle interconnecting said pair of vertical side members, said handle oriented in a substantially horizontal manner and positioned above said lower frame, said handle vertically adjustable along said vertical side members, said handle having a first bracket located at a first end of said handle and a second bracket located at a second end of said handle, said brackets connecting said handle to said pair of said vertical side members, said brackets each having a orifice for receiving one of said spring loaded pins therethrough to lock said bracket to a specific location on said vertical side member; and
at least one removable armrest connected to one of said vertical side members of said upper frame, said removable armrest rotatably adjustable about said vertical side member and adjustable along said vertical side member, said removable armrest having a clam-shaped body with an interior surface for engaging a surface of said horizontal member, said clam-shaped body including a set of corresponding lips, and further including a nut and bolt securement system located on said lip of clam-shaped body.

14. The MRI/CAT scan patient arm support assembly of claim 13 wherein said at least one cross member of said lower frame is curved-shaped to conform to a curve shaped surface of a MRI/CAT scan bed.

15. The MRI/CAT scan patient arm support assembly of claim 13 wherein each of said vertical side members include said removable armrest connected thereto.

16. The MRI/CAT scan patient arm support assembly of claim 13 wherein said lower frame includes a removable head foam block pillow having a patient headrest region fixedly secured to said lower frame by a hook and loop fasteners system.

17. The MRI/CAT scan patient arm support assembly of claim 13 wherein said lower frame includes a headrest-receiving member extending from said cross member.

18. The MRI/CAT scan patient arm support assembly of claim 13 including a hinge means pivotally connecting said pair of horizontal side members of said lower frame to said pair of vertical side members of said upper frame; and
an adjustment means interconnecting said lower frame with said upper frame, said adjustment means adapted to secure said upper frame in a plurality of angularly positions relative said lower frame.

19. A MRI/CAT scan patient arm support assembly comprising:
a lower frame including a pair of horizontal side members positioned parallel to each other;
at least one cross member connecting said pair of horizontal side members, said pair of horizontal side members and said at least one cross member forming a curved shaped MRI/CAT scan bed-engaging surface;
an upper frame connected to and extending angularly upward from said lower frame, said upper frame having a pair of vertical side members positioned parallel to each other, said pair of vertical side members each including a plurality of spring loaded pins extending from and spaced along said vertical side members;
a handle interconnecting said pair of vertical side members, said handle oriented in a substantially horizontal manner and positioned above said lower frame, said handle vertically adjustable along said vertical side members, said handle having a first bracket located at a first end of said handle and a second bracket located at a second end of said handle, said brackets connecting said handle to said pair of said vertical side members, said brackets each having a orifice for receiving one of said spring loaded pins therethrough to lock said bracket to a specific location on said vertical side member;
a hinge means pivotally connecting said pair of horizontal side members of said lower frame to said pair of vertical side members of said upper frame;
an adjustment means interconnecting said lower frame with said upper frame, said adjustment means adapted to secure said upper frame in a plurality of angularly positions relative said lower frame; and
a removable armrest connected to each of said vertical side members of said upper frame, said removable armrests rotatably adjustable about said vertical side members and adjustable along said vertical side members, said removable armrests each having a clam-shaped body with an interior surface for engaging a surface of said horizontal member, said clam-shaped body including a set of corresponding lips, and further including a nut and bolt securement system located on said lip of clam-shaped body.

20. The MRI/CAT scan patient arm support assembly of claim 19 wherein said lower frame includes a headrest-receiving member extending from said cross member.
